(19) United States
(12) Patent Application Publication
(10) Pub. No.: US 2002/0009772 A1
(43) Pub. Date: Jan. 24, 2002

(54) NOVEL HUMAN CALCIUM CHANNELS AND RELATED PROBES, CELL LINES AND MEHTODS

(76) Inventor: TERRY P. SNUTCH, VANCOUVER (CA)

Correspondence Address: KATE H. MURASHIGE, MORRISON & FOERSTER LLP, 3811 VALLEY CENTRE DRIVE, SUITE 500, SAN DIEGO, CA, 92130-2071, US

(21) Appl. No.: 09/030,482

(22) Filed: Feb. 25, 1998

Related U.S. Application Data
(60) Provisional application No. 60/039,204, filed on Feb. 28, 1997.

(51) Int. Cl. G01N 33/53; C07H 21/04; C12P 21/06; C12N 15/00; C12N 15/09; C12N 15/63; C12N 15/70; C12N 15/74; C12N 5/00; C12N 5/02

(52) U.S. Cl. 435/69.1; 536/23.5; 435/320.1; 435/325; 435/7.1

(57) ABSTRACT

Partial sequences for a novel mammalian (human and rat sequences identified) calcium channel subunit which we have labeled as the α_{1I} subunit, and an additional novel human calcium channel which we have labeled as the α_{1H} subunit are provided. Knowledge of the sequence of these two calcium channels permits the localization and recovery of the complete sequence from human cells, and the development of cell lines which express the novel calcium channels of the invention. These cells may be used for identifying compounds capable of acting as agonists or antagonists to the calcium channels.

This application is a regular application filed under 35 USC § 111 (a), claiming priority from U.S. Provisional Patent Application No. 60/039,204 filed Feb. 28, 1997.

### TECHNICAL FIELD

The present invention relates to novel human calcium channel compositions, and to the expression of these compositions in cell lines for use in evaluating calcium channel function.

### BACKGROUND OF THE INVENTION

The rapid entry of calcium into cells is mediated by a class of proteins called voltage-gated calcium channels. Calcium channels are a heterogeneous class of molecules that respond to depolarization by opening a calcium-selective pore through the plasma membrane. The entry of calcium into cells mediates a wide variety of cellular and physiological responses including excitation-contraction coupling, hormone secretion and gene expression. In neurons, calcium entry directly affects membrane potential and contributes to electrical properties such as excitability, repetitive firing patterns and pacemaker activity. Miller, R. J. (1987) Multiple calcium channels and neuronal function. Science 235:46-52. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter. Calcium entry also plays a role in neurite outgrowth and growth cone migration in developing neurons and has been implicated in long-term changes in neuronal activity. In addition to the variety of normal physiological functions mediated by calcium channels, they are also implicated in a number of human disorders. Recently, mutations identified in human and mouse calcium channel genes have been found to account for several disorders including, familial hemiplegic migraine, episodic ataxia type 2, cerebellar ataxia, absence epilepsy and seizures. Fletcher, C. F., Lutz, C. M., O'Sullivan, T. N., Shaughnessy, Jr., J. D., Hawkes, R., Frankel, W. N., Copeland, N. G. and Jenkins, N. A. (1996) Absence epilepsy in tottering mutant mice is associated with calcium channel defects. Cell 87:607-617; Burgess, D. L., Jones, J. M., Meiser, M. H. and Noebels, J. L. (1997) Mutation of the Ca2+channel β subunit gene Cchb4 is associated with ataxia and seizures in the lethargic (lh) mouse. Cell 88:385-392; Ophoff, R. A., Terwindt, G. M., Vergouwe, M. N., van Eijk, R., Oefner, P. J., Hoffman, S. M. G., Lamerdin, J. E., Mohrenweiser, H. W., Bulman, D. E., Ferrari, M., Haan, J., Lindhout, D., van Ommen, G. -J. B., Hofker, M. H., Ferrari, M. D. and Frants, R. R. (1996) Familial hemiplegic migraine and episodic ataxia type-2 are caused by mutations in the Ca2+channel gene CACNL1A4. cell 87:543-552; Zhuchenko, O., Bailey, J., Bonnen, P., Ashizawa, T., Stockton, D. W., Amos, C., Dobyns, W. B., Subramony, S. H., Zogbhbi, H. Y. and Lee, C. C. (1997) Autosomal dominat cerebellar ataxia (SCA6) associated with the small polyglutamine expansions in the α1A-voltage-dependent calcium channel. Nature Genetics 15:62-69. The clinical treatment of some disorders has been aided by the development of therapeutic calcium channel antagonists. Janis, R. J and Triggle, D. J. (1991) In Calcium Channels: Their Properties, Functions, Regulation and Clinical Relevance. CRC Press, London.

Native calcium channels have been classified by their electrophysiological and pharmacological properties as T, L, N, P and Q types (for reviews see McCleskey, E. W. and Schroeder, J. E. (1991) Functional properties of voltage-dependent calcium chnanels. Curr. Topics Membr. 39: 295-326, and Dunlap, K., Luebke, J. I. and Turner, T. J. (1995) Exocytotic Ca²⁺ channels in mammalian central neurons. Trends Neurosci. 18:89-98.). T-type (or low voltage-activated) channels describe a broad class of molecules that transiently activate at negative potentials and are highly sensitive to changes in resting potential. The L, N, P and Q-type channels activate at more positive potentials and display diverse kinetics and voltage-dependent properties. There is some overlap in biophysical properties of the high voltage-activated channels, consequently pharmacological profiles are useful to further distinguish them. L-type channels are sensitive to dihydropyridine (DHP) agonists and antagonists, N-type channels are blocked by the Conus geographus peptide toxin, ω-conotoxin GVIA, and P-type channels are blocked by the peptide ω-agatoxin IVA from the venom of the funnel web spider, Agelenopsis aperta. A fourth type of high voltage-activated Ca channel (Q-type) has been described, although whether the Q- and P-type channels are distinct molecular entities is controversial (Sather, W. A., Tanabe, T., Zhang, J. -F., Mori, Y., Adams, M. E., and Tsien, R. W. (1993) Distinctive biophysical and pharmacological properties of class A (B 1) calcium channel α1 subunits. Neuron 11: 291-303; Stea, A., Tomlinson, W. J., Soong, T. W., Bourinet, E., Dubel, S. J., Vincent, S. R and Snutch, T. P. (1994) Localization and functional properties of a rat brain α1A calcium channel reflect similarities to neuronal Q- and P-type channels. PNAS 91: 10576-10580.). Several types of calcium conductances do not fall neatly into any of the above categories and there is variability of properties even within a category suggesting that additional calcium channels subtypes remain to be classified.

Biochemical analyses show that neuronal calcium channels are heterooligomeric complexes consisting of three distinct subunits (α₁, α₂δ and β) (reveiwed by De Waard, M., Gurnett, C. A. and Campbell, K. P. (1997) In Ion Channels, Volume 4, edited by Narahashi, T. Plenum Press, New York). The α₁ subunit is the major pore-forming subunit and contains the voltage sensor and binding sites for calcium channel antagonists. The mainly extracellular α₂ is disulphide-linked to the transmembrane δ subunit and both are derived from the same gene and are proteolytically cleaved in vivo. The β subunit is a non-glycosylated, hydrophilic protein with a high affinity of binding to a cytoplasmic region of the α₁ subunit. A fourth subunit, γ, is unique to L-type Ca channels expressed in skeletal muscle T-tubules. The isolation and characterization of γ-subunit-encoding cDNAs is described in U.S. Pat. No. 5,386,025 which is incorporated herein by reference.

Molecular cloning has revealed the cDNA and corresponding amino acid sequences of six different types of α₁ subunits (α_{1A}, α_{1B}, α_{1C}, α_{1D}, α_{1E} and α_{1S}) and four types of β subunits (β₁, β₂, β₃ and β₄) (reviewed in Stea, A., Soong, T. W. and Snutch, T. P. (1994) Voltage-gated calcium channels. PCT Patent Publication WO 95/04144, which is incorporated herein by reference, discloses the sequence and expression of α_{1E} calcium channel subunits. In Handbook of Receptors and Channels. Edited by R. A. North, CRC Press.).

The different classes of (α1 and β subunits have been identified in different animals including, rat, rabbit and human and share a significant degree of amino acid conservation across species (for examples see: Castellano, A., Wei, X., Bimbaumer, L., and Perez-Reyes, E. (1993) Cloning and expression of a third calcium channel β subunit. J. Biol. Chem. 268: 3450-3455; Castellano, A., Wei, X., Bimbaumer, L., and Perez-Reyes, E. (1993) Cloning and expression of a neuronal calcium channel β subunit. J. Biol. Chem. 268: 12359-12366; Dubel, S. J., Starr, T. V. B., Hell, J., Ahlijanian, M. K., Enyeart, J. J., Catterall, W. A., and Snutch, T. P. (1992). Molecular cloning of the α₁ subunit of an co-conotoxin-sensitive calcium channel. Proc. Natl. Acad. Sci. USA 89: 5058-5062; Fujita, Y., Mynlieff, M., Dirksen, R. T., Kim, M., Niidome, T., Nakai, J., Friedrich, T., Iwabe, N., Miyata, T., Furuichi, T., Furutama, D., Mikoshiba, K., Mori, Y., and Beam, K. G. (1993) Primary structure and functional expression of the ω-conotoxin-sensitive N-type calcium channel from rabbit brain. Neuron 10: 585-598; Mikami, A., Imoto, K., Tanabe, T., Niidome, T., Mori, Y., Takeshima, H., Narumiya, S., and Numa, S. (1989). Primary structure and functional expression of the cardiac dihydropyridine-sensitive calcium channel. Nature 340: 230-233; Mori, Y., Friedrich, T., Kim, M. -S., Mikami, A., Nakai, J., Ruth, P., Bosse, E., Hofmann, F., Flockerzi, V., Furuichi, T., Mikoshiba, K., Imoto, K., Tanabe, T., and Numa, S. (1991) Primary structure and functional expression from complementary DNA of a brain calcium channel. Nature 350: 398-402; Perez-Reyes, E., Castellano, A., Kim, H. S., Bertrand, P., Baggstrom, E., Lacerda, A. E., Wei, X., and Bimbaumer, L. (1992). Cloning and expression of a cardiac/brain β subunit of the L-type calcium channel. S. Biol. Chem. 267: 1792-1797; Pragnell, M., Sakamoto, J., Jay, S. D., and Campbell, K. P. (1991). Cloning and tissue-specific expression of the brain calcium channel β-subunit. FEBS Lett. 291: 253-258; Snutch, T. P., Tomlinson, W. J., Leonard, J. P., and Gilbert, M. M. (1991) Distinct calcium channels are generated by alternative splicing and are differentially expressed in the mammalian CNS. Neuron 7: 45-57; Soong, T. W., Stea, A., Hodson, C. D., Dubel, S. J., Vincent, S. R., and Snutch, T. P. (1993) Structure and functional expression of a member of the low voltage-activated calcium channel family. Science 260: 1133-1136; Tomlinson, W. J., Stea, A., Bourinet, E., Charnet, P., Nargeot, J., and Snutch, T. P. (1993) Functional properties of a neuronal class C L-type channel. Neuropharmacology 32: 1117-1126; Williams, M. E., Feldman, D. H., McCue, A. F., Brenner, R., Velicelebi, G., Ellis, S. B., and Harpold, M. M. (1992) Structure and functional expression of α1, α2, and β subunits of a novel human neuronal calcium channel subtype. Neuron 8: 71-84; Williams, M. E., Brust, P. F., Feldman, D. H., Patthi, S., Simerson, S., Maroufi, A., McCue, A. F., Velicelebi, G., Ellis, S. B., and Harpold, M. (1992) Structure and functional expression of an Co-conotoxin-sensitive human N-type calcium channel. Science 257: 389-395.

In some expression systems the α₁ subunits alone can form functional calcium channels although their electrophysiological and pharmacological properties can be differentially modulated by coexpression with any of the four β subunits. Until recently, the reported modulatory affects of β subunit coexpression were to mainly alter kinetic and voltage-dependent properties. More recently it has been shown that β subunits also play crucial roles in modulating channel activity by protein kinase A, protein kinase C and direct G-protein interaction. (Bourinet, E., Charnet, P., Tomlinson, W. J., Stea, A., Snutch, T. P. and Nargeot, J. (1994) Voltage-dependent facilitation of a neuronal α1C L-type calcium channel. EMBO J. 13: 5032-5039; Stea, A., Soong, T. W. and Snutch, T. P. (1995) Determinants of PKC-dependent modulation of a family of neuronal calcium channels. Neuron 15:929-940; Bourinet, E., Soong, T. W., Stea, A. and Snutch, T. P. (1996) Determinants of the G-protein-dependent opioid modulation of neuronal calcium channels. Proc. Natl. Acad. Sci. (USA) 93: 1486-1491.)

The electrophysiological and pharmacological properties of the calcium channels cloned to date can be summarized as shown in Table 1. While the cloned α₁ subunits identified to date correspond to several of the calcium channels found in cells, they do not account for all types of calcium conductances described in native cells. For example, they do not account for the various properties described for the heterogenous family described as T-type calcium channels. Furthermore, they do not account for novel calcium channels described in cerebellar granule cells or other types of cells. (Forti, L. and Pietrobon, D. (1993) Functional diversity of L-type calcium channels in rat cerebellar neurons. Neuron 10: 437-450; Tottene, A., Moretti, A., Pietrobon, A. 1996. Functional diversity of P-type and R-type calcium channels in rat cerebellar neurons. J. Neurosci. 16: 6353-6363).

Because of the importance of calcium channels in cellular metabolism and human disease, it would be desirable to identify the remaining classes of α₁ subunits, and to develop expression systems for these subunits which would permit the study and characterization of these calcium channels, including the study of pharmacological modulators of calcium channel function. Thus, it is an object of the present invention to provide heretofor undisclosed calcium channels having novel α₁ subunits, including cell lines expressing these new calcium channels. It is a further object of the present invention to provide a method for testing these novel calcium channels using such cell lines.

TABLE 1

| | ω-conotoxin | 1,4- | | ω-agatoxin | ω-conotoxin | native |
|---|---|---|---|---|---|---|
| | GVIA | dihydropyridines | cadmium | IVA | MVIIC | Ca²⁺ channel type |
| α_{1A} | — | — | ✓ | ✓ | ✓ | P/Q-type |
| α_{1B} | ✓ | — | ✓ | — | ✓ | N-type |
| α_{1C} | — | ✓ | ✓ | — | — | L-type |
| α_{1D} | — | ✓ | ✓ | — | — | L-type |
| α_{1E} | — | — | ✓ | — | — | novel |
| α_{1S} | — | ✓ | ✓ | — | — | L-type |

### SUMMARY OF THE INVENTION

The present invention provides partial sequences for a novel mammalian (human and rat sequences identified) calcium channel subunit which we have labeled as the α_{1I} subunit, and an additional novel human calcium channel which we have labeled as the α_{1H} subunit. This knowledge of the sequence of these two calcium channels permits the localization and recovery of the complete sequence from human cells, and the development of cell lines which express the novel calcium channels of the invention. These cells may be used for identifying compounds capable of acting as agonists or antagonists to the calcium channels.

### BRIEF DESCRIPTION OF THE DRAWINGS

FIG. 1 shows aligned amino acid sequences for the C. elegans C54D2.5 α₁ calcium channel subunit and initially identified portions of the calcium channel subunits of the invention.

### DESCRIPTION OF THE INVENTION

The present invention includes the following aspects for which protection is sought:

(a) novel human calcium channel subunits and DNA fragments encoding such subunits. It will be appreciated that polymorphic variations may be made or may exist in the DNA of some individuals leading to minor deviations in the DNA or amino acids sequences from those shown which do not lead to any substantial alteration in the function of the calcium channel. Such variations, including variations which lead to substitutions of amino acids having similar properties are considered to be within the scope of the present invention.

(b) polynucleotide sequences useful as probes in screening human cDNA libraries for genes encoding these novel calcium channel subunits. These probes can also be used in histological assay to determine the tissue distribution of the novel calcium channel subunits.

(c) eukaryotic cell lines expressing the novel calcium channel subunits. These cell lines can be used to evaluate compounds as pharmacological modifiers of the function of the novel calcium channel subunits.

(d) a method for evaluating compounds as pharmacological modifiers of the function of the novel calcium channel subunits using the cell lines expressing those subunits alone or in combination with other calcium channel subunits.

Further, since defects in the novel calcium channel subunits may be associated with a human genetic disease including, but not limited to; epilepsy, migraine, ataxia, schizophrenia, hypertension, arrhythmia, angina, depression, small lung carcinoma, Lambert-Eaton syndrome, characterization of such associations and ultimately diagnosis of associated diseases can be carried out with probes which bind to the wild-type or defective forms of the novel calcium channels.

In accordance with the present invention, we have identified human DNA sequences which code for novel calcium channel α₁ subunits. These subunits are believed to represent two new types of α₁ subunits of human voltage-dependent calcium channels which have been designated as type α_{1I} and type α_{1H}.

The novel α₁ subunits of the invention were identified by screening the C. elegans genomic DNA sequence data base for sequences homologous to previously identified mammalian calcium channel α₁ subunits. Specifically, the following twelve mammalian α₁ subunit sequences were used to screen the C. elegans genomic data bank:

rat brain α_{1A}: GTCAAAACTC AGGCCTTCTA CTGG SEQ ID. No. 1

rat brain α_{1A}: AACGTGTTCT TGGCTATCGC GGTG SEQ ID. No. 2

rat brain α_{1B}: GTGAAAGCAC AGAGCTTCTA CTGG SEQ ID. No. 3

rat brain α_{1B}: AACGTTTTCT TGGCCATTGC TGTG SEQ ID. No. 4

rat brain α_{1C}: GTTAAATCCA ACGTCTTCTA CTGG SEQ ID. No. 5

rat brain α_{1C}: AATGTGTTCT TGGCCATTGC GGTG SEQ ID. No. 6

rat brain α_{1D}: GTGAAGTCTG TCACGTTTTA CTGG SEQ ID. No. 7

rat brain α_{1D}: AAGCTCTTCT TGGCCATTGC TGTA SEQ ID. No. 8

rat brain α_{1E}: GTCAAGTCGC AAGTGTTCTA CTGG SEQ ID. No. 9

rat brain α_{1E}: AATGTATTCT TGGCTATCGC TGTG SEQ ID. No. 10

rat brain consensus #1: ATCTAYGCYR TSATYGGSAT G SEQ ID. No. 11

rat brain consensus #2: ATGGACAAYT TYGASTAYTC SEQ ID. No. 12

This search identified four distinct C. elegans cosmids that contain open reading frames (coding regions) that exhibit homology to mammalian calcium channel α₁ subunits:

cosmid and reading frame T02C5.5

cosmid and reading frame C48A7.1

cosmid and reading frame C54D2.5

cosmid and reading frame C27F2.3

Examination of the four C. elegans cosmid sequences by phylogeny analysis shows that two of these, T02C5.5 and C48A7.1, correspond closely with previously identified mammalian α₁ subunits. T02C5.5 appears to be an ancestral member related to the mammalian α_{1A}, α_{1B} and α_{1E} subunits. C48A7.1 appears to be an ancestral member related to the mammalian L-type channels encoded by α_{1C}, α_{1D} and α_{1S}. In contrast, the C. elegans cosmids C54D2.5 and C27F2.3 identify novel types of calcium channel α₁ subunits distinct from the other mammalian subtypes.

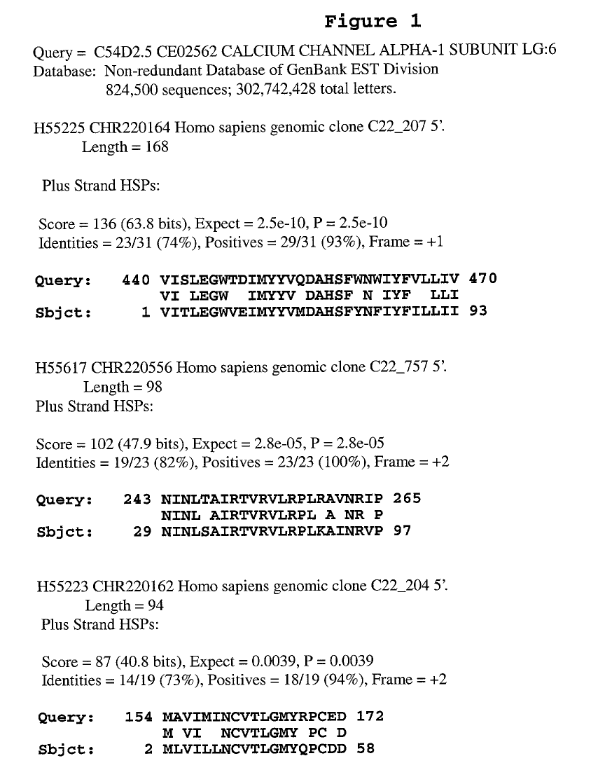

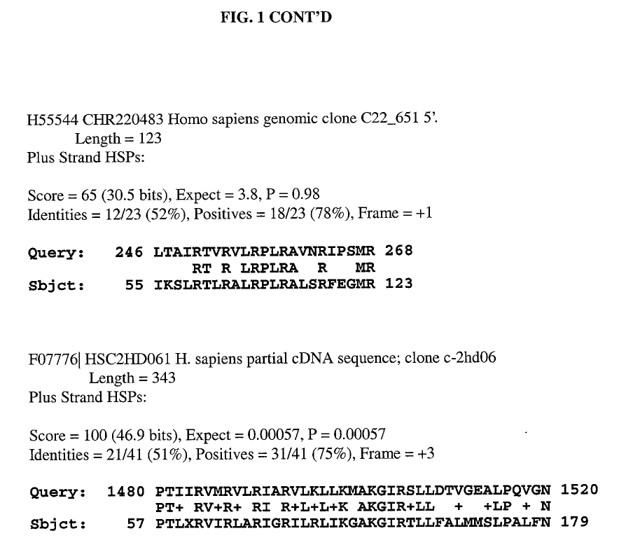

Mammalian counterparts of the C. elegans calcium channel α₁ subunit encoded by C54D2.5 were identified by screening of the GenBank expressed sequence tag (EST) data bank. This analysis identified a total of 13 mammalian sequences that exhibit some degree of DNA sequence and amino acid identity to C54D2.5, of which 8 are human sequences. (Table 2) Three of these sequences appear unlikely to encode novel calcium channel subunits because they either exhibit a significant degree of homology to previously identified mammalian α₁ subunits (clones H06096 and H14053) or exhibit homology in a region not considered to be diagnostic of calcium channel α₁ subunits specifically as opposed to other types of ion channel molecules in general (clone D20469). The five remaining sequences (H55225, H55617, H55223, H55544, and F07776), however, are believed to encode two previously unidentified calcium channel α₁ subunits because the degree of amino acid identity closely matches that of known calcium channel subunits in conserved regions but is sufficiently different to indicate that they do not encode previously identified mammalian calcium channel α₁ subunits α_{1A}, α_{1B}, α_{1C}, α_{1D}, α_{1E}, or α_{1S}. The expected amino acid sequence closely matches but is not identical to amino acid sequences in these known calcium channel subunits. The aligned amino acids sequences are shown in FIG. 1.

TABLE 2
Query = C54D2.5 CE02562 CALCIUM CHANNEL ALPHA-1 SUBUNIT LG:6

| | |
|---|---|
| Database: | Non-redundant Database of GenBank EST Division |
| | 824,500 sequences; 302,742,428 total letter |

| Sequences producing High-scoring Segment Pairs: | | Frame | Score | P(N) |
|---|---|---|---|---|
| gb\|AA183990\|AA183990 | ms53e02.r1 Life Tech mouse embry . . . | +1 | 108 | 1.8e-24 |
| gb\|H55225\|H55225 | CHR220164 Homo sapiens genomic c . . . | +1 | 136 | 2.5e-10 |
| db\|D68412\|CELK131B1F | C.elegans cDNA clone yk131b1:5 . . . | +3 | 117 | 1.7e-06 |
| gb\|R75128\|R75128 | MDB1075 Mouse brain, Stratagene . . . | +3 | 113 | 7.2e-06 |
| gb\|H55617\|H55617 | CHR220556 Homo sapiens genomic c . . . | +2 | 102 | 2.8e-05 |
| emb\|FO7776\|HSC2HD061 | H. sapiens partial CDNA sequence . . . | +3 | 100 | 0.00057 |
| gb\|W76774\|W76774 | me84e08.rl Scares mouse embryo N . . . | +2 | 98 | 0.0012 |
| gb\|H06096\|E06096 | y177e01.rl Homo sapiens cDNA clo . . . | +3 | 98 | 0.0015 |
| gb\|H14053\|H14053 | ym65d10.r1 Homo sapiens cONA clo . . . | +2 | 91 | 0.0036 |
| gb\|H55223\|H55223 | CHR220162 Homo sapiens genoinic c . . . | +2 | 87 | 0.0039 |
| dbj\|D35703\|CELK024D9F | C. elegans cDNA clone yk24d9: 5′. . . | +3 | 74 | 0.046 |
| dbj\|D20469\|HUMGS01443 | Human HL60 3′ directed MboI cDNA, . . . | −2 | 66 | 0.91 |
| gb\|H55544\|H55544 | CHR220483 Homo sapiens genomic c . . . | +1 | 65 | 0.98 |

Four of the five sequences (H55225, H55617, H55223, and H55544) are found on human chromosome 22, and are now believed to all be part of the same gene encoding the novel human calcium channel subunit all. The fifth sequence, F07776 is apparently distinct and associated with a further novel human calcium channel subunit designated O11H.

The sequences of the five selected sequences and the references from which they are taken are given as follows:

H55225 SOURCE human clone=C22_{—}207 primer=T3 library=Chromosome 22 exon Trofatter, et al., Genome Res. 5 (3): 214-224 (1995) SEQ ID No. 13 1 GTGATCACTC TGGAAGGCTG GGTGGAGATC ATGTACTACG TGATGGATGC TCACTCCTTC 61 TACAACTTCA TCTACTTCAT CCTGCTTATC ATACCCCTCT TGCCTTGCAC CCCATATGGT 121 CTTCCCAGAG TGAGCTCATC CACCTCGTCA TGCCTGACTC GACGTTCA

1155617 SOURCE human clone=C22_{—}757 primer=T3 library=Chromosome 22 exon Trofatter, et al., Genome Res. 5 (3): 214-224 (1995) SEQ ID No. 14 1 GATGGTCGAG TACTCCCTGG ACCTTCAGAA CATCAALCCTG TCAGCCATCC GCACCGTGCG 61 CGTCCTGAGG CCCCTCAAAG CCATCAACCG CGTGCCCA

H55223 SOURCE human clone=C22_{—}204 primer=T3 library=Chromosome 22 exon Trofatter, et al, Genome Res. 5 (3): 214-224 (1995) SEQ ID No. 15 1 CATGCTGGTG ATCCTGCTGA ACTGCGTGAC ACTTGGCATG TACCAGCCGT GCGACGACAT 61 GGACTGCCTG TCCGACCGCT GCAAGATCCT GCAG

H55544 SOURCE human clone=C22_{—}651 primer=T3 library=Chromosome 22 exon Trofatter, et al, Genome Res. 5 (3): 214-224 (1995) SEQ ID No. 16 1 GTATCTCTGG TTACTTTAGT AGCCAACACT CTTGGCTACT CAGACCTTGG TCCCATTAA 61 TCCCTGCGAA CCTTGAGAGC ACTAAGACCT CTAAGAGCTT TGTCTAGATT TGAAGTAATG 121 AGG

F07776 SOURCE human. Submitted (Jan. 19, 1995) Genethon, B. P. 60, 91002 Evry Cedex France and Genetique Moleculaire et Biologie du developpement, CNRS UPR420 B. P. 8, 94801 Villejuif Cedex France E-mail: genexpress@genethon.fr SEQ ID No. 17 1 TTCTCTCCAT TGTAGGAATG TTTCTGGCTG AACTGATAGA AAAGTATTTT GTGTGCCCTA 61 CCCTGTTNCG AGTGATCCGT CTTGCCAGGA TTGGCCGAAT CCTACGTCTG ATCAAAGGAG 121 CAAAGGGGAT CCGCACGCTG CTCTTTGCTT TGATGATGTC CCTTCCTGCG TTGTTTAACA 181 TCGGNCTCCT TCTTTTCCTG GTCATGTTCA TCTACGNCAT CTTTGGGATG TCCAATTTTG 241 CCTATGTTAA GAGGGAAGTT GGGATCGATG ACATGTTNAN CTTTGAGACC TTTGGCAACA 301 GCATGATCTG CCTGTTCCAA ATTACAACCT CTGCTGGCTG GGA

A search of the Sanger Genome Sequencing Center (Cambridge, U.K.) and the Washington University Genome Sequencing Center (St. Louis. Mo.) sequences in progress revealed a Bacterial Artificial Chromosome (BAC) sequence (bK206c7) that contained matches to the C. elegans cosmid open reading frame, C54D2.5, and to the four human chromosome 22 ESTs, H55225, H55617, H55223,H55544. The C. elegans C54D2.5 cosmid sequence and the human EST sequences were then used to compare the translation of the bK206c7 BAC genomic sequence in all 6 reading frames. The analysis was performed using the graphical program Dotter (Eric Sohnhammer, NCBI). The analysis revealed a series of potential coding regions on one strand of the bK206c7 BAC sequence. These were subsequently translated in all 3 reading frames and the potential splice junctions identified. The translated sequence of this longer DNA fragment which is part of the human α_{1I} subunit gene is given by Seq. ID No. 18.

Using the sequence information from the five EST's, a full length gene can be recovered using any of several techniques. Polynucleotide probes having a sequence which corresponds to or hybridizes with the EST sequences or a distinctive portion thereof (for example oligonucleotide probes having a length of 18 to 100 nucleotides) can be used to probe a human cDNA library for identification of the full length DNA encoding the α_{1I} and α_{1H} subunits. The process of identifying cDNAs of interest using defined probes is well known in the art and is, for example, described in International Patent Publication No. WO95/04144, which is incorporated herein by reference. This process generally involves screening bacterial hosts (e.g. E. coli) harboring the library plasmids or infected with recombinant lambda phage with labeled probes, e.g. radiolabeled with ³²p, and selection of colonies or phage which bind the labeled probe. Each selected colony or phage is grown up, and the plasmids are recovered. Human cDNAs are recovered from the plasmids by restriction digestion, or can be amplified, for example by PCR. The recovered cDNA can be sequenced, and the position of the calcium channel subunit-encoding region further refined, although neither process is not necessary to the further use of the cDNA to produce cell lines expressing the novel calcium channel subunits.

Longer portions of DNA-encoding the novel calcium channel subunits of the invention can also be recovered by PCR cloning techniques using primers corresponding to or based upon the EST sequences. Using this technique to identify relevant sequences within a human brain total RNA preparation confirmed that the novel α_{1I} calcium channel subunit is present in human brain. Subcloning of the 567 nt PCR product and subsequent sequencing thereof showed that this product corresponds to the derived sequence form the bK206c7 BAC genomic sequence. The nucleotide sequence is given as SEQ ID No. 19. The same experiment was performed using a rat brain RNA preparation and resulted in recovery of a substantially identical PCR product. (SEQ ID. NO. 20). The protein encoded by the rat PCR product is 96% identical to the human PCR product.

These sequences, which presumably encode a partial subunit can be used as a basis for constructing full length human or rat a,, clones. Briefly, the subcloned α_{1I} PCR product is radiolabeled by random hexamer priming according to standard methods (See, Sambrook, J., Fritsch, E. F. and Maniatis, T. (1989) Molecular Cloning, A Laboratory Manual. Cold Spring Harbor Press) and used to screen commercial human brain cDNA libraries (Stratagene, La Jolla, Calif.). The screening of cDNA libraries follows standard methods and includes such protocols as infecting bacteria with recombinant lambda phage, immobilizing lambda DNA to nitrocellulose filters and screening under medium hybridization stringency conditions with radiolabeled probe. cDNA clones homologous to the probe are identified by autoradiography. Positive clones are purified by sequential rounds of screening.

Following this protocol, most purified cDNA's are likely to be partial sequence clones due the nature of the cDNA library synthesis. Full length clones are constructed from cDNA's which overlap in DNA sequence. Restriction enzyme sites which overlap between cDNAs are used to ligate the individual cDNA's to generate a full-length cDNA. For subsequent heterologous expression, the full-length cDNA is subcloned directly into an appropriate vertebrate expression vector, such as pcDNA-3 (Invitrogen, San Diego, Calif.) in which expression of the cDNA is under the control of a promoter such as the CMV major intermediate early promoter/enhancer. Other suitable expression vectors include, for example, pMT2, pRCICMV, pcDNA3.1 and pCEP4.

Once the full length cDNA is cloned into an expression vector, the vector is then transfected into a host cell for expression. Suitable host cells include Xenopus oocytes or mammalian cells such as human embryonic kidney cells as described in International Patent Publication No. WO 96/39512 which is incorporated herein by reference and Ltk cells as described in U.S. Pat. No. 5,386,025 which is incorporated herein by reference. Transfection into host cells may be accomplished by microinjection, lipofection, glycerol shock, electroporation calcium phosphate or particle-mediated gene transfer. The vector may also be transfected into host cells to provide coexpression of the novel α₁ subunits with a β and/or an α₂δ subunit.

The resulting cell lines expressing functional calcium channels including the novel α₁ subunits of the invention can be used test compounds for pharmacological activity with respect to these calcium channels. Thus, the cell lines are useful for screening compounds for pharmaceutical utility. Such screening can be carried out using several available methods for evaluation of the interaction, if any, between the test compound and the calcium channel. One such method involves the binding of radiolabeled agents that interact with the calcium channel and subsequent analysis of equilibrium binding measurements including but not limited to, on rates, off rates, K_{d} values and competitive binding by other molecules. Another such method involves the screening for the effects of compounds by electrophysiological assay whereby individual cells are impaled with a microelectrode and currents through the calcium channel are recorded before and after application of the compound of interest. Another method, high-throughput spectrophotometric assay, utilizes the loading the cell lines with a fluorescent dye sensitive to intracellular calcium concentration and subsequent examination of the effects of compounds on the ability of depolarization by potassium chloride or other means to alter intracellular calcium levels. Compounds to be tested as agonists or antagonists of the novel (α_{1I} and α_{1H} calcium channel subunits are combined with cells that are stably or transiently transformed with a DNA sequence encoding the α_{1I} or α_{1H} calcium channel subunits of the invention and monitored using one of these techniques.

DNA fragments with sequences given by SEQ ID Nos. 13-19 may also be used for mapping the distribution of α_{1I} and α_{1H} calcium channel subunits within a tissue sample. This method follows normal histological procedures using a nucleic acid probe, and generally involves the steps of exposing the tissue to a reagent comprising a directly or indirectly detectable label coupled to a selected DNA fragment, and detecting reagent that has bound to the tissue. Suitable labels include fluorescent labels, enzyme labels, chromophores and radio-labels.

### EXAMPLE 1

In order to isolate novel human calcium channel α₁ subunits using standard molecular cloning protocols, synthetic DNA probes are prepared, radiolabeled with ³²P and utilized to screen human cDNA libraries commercially available in lambda phage vectors (Stratagene, La Jolla, Calif.) based on the human DNA sequences for H55225, H55617, H55223, H55544 and F07776. DNA fragments with the sequence of sequence ID NOs 18 and 19 may also be used for this purpose. Positive phage are purified through several rounds of screening involving immobilizing the phage DNA on nitrocellulose filters, hybridizing with the radiolabeled probe, washing off of excess probe and then selection of clones by autoradiography. Clones identified by this approach are expected to be partial length clones due to the nature of cDNA library synthesis and several rounds of screening for each calcium channel type may be necessary to obtain full-length clones.

To characterize the clones, double stranded plasmid DNA is prepared from the identified clones and the sequences are determined using 35S dATP, Sequenase and standard gel electrophoresis methods. Regions of similarity and regions of overlap are determined by comparison of each cDNA sequence.

Full-length clones are constructed by ligating overlapping cDNA fragments together at common restriction enzyme sites. The full-length clones are subsequently inserted into vectors suitable for expression in vertebrate cells (e.g. pMT2, pRC/CMV, pcDNA3.1, pCEP4, pREP7) by ligation into restriction sites in the vector polylinker region which is downstream of the promoter used to direct cDNA expression.

DNA encoding the novel calcium channels can be stably or transiently introduced into eukaryotic cells (e.g. human embryonic kidney, mouse L cells, chinese hamster ovary, etc) by any number of available standard methods. Stable transfection is achieved by growing the cells under conditions that promote growth of cells expressing a marker gene which is contained in the expression vector (e.g. dihydrofolate reductase, thymidine kinase, or the like). The heterologous DNA encoding the human calcium channel may be integrated into the genome or may be maintained as an episomal element.

Expression of the human calcium channel in transfected cells may monitored by any number of techniques, including Northern blot for RNA analysis, Southern blot for cDNA detection, electrophysiological assay for calcium channel function, the binding of radiolabeled agents thought to interact with the calcium channel, and fluorescent assay of dyes sensitive to intracellular calcium concentration.

### EXAMPLE 2

### Heterologous Expression of Human α_{1I} Calcium Channels in Cells

A. Transient Transfection in Mammalian Cells

Host cells, such as human embryonic kidney cells, HEK 293 (ATCC# CRL 1573) are grown in standard DMEM medium supplemented with 2 mM glutamine and 10% fetal bovine serum. HEK 293 cells are transfected by a standard calcium-phosphate-DNA co-precipitation method using the full-length human α_{1I} calcium channel cDNA in a vertebrate expression vector (for example see Current protocols in Molecular Biology). The human α_{1I} calcium channel cDNA may be transfected alone or in combination with other cloned subunits for mammalian calcium channels, such as α2δ and β subunits, and also with clones for marker proteins such the jellyfish green fluorescent protein.

Electrophysiological Recording: After an incubation period of from 24 to 72 hrs the culture medium is removed and replaced with external recording solution (see below). Whole cell patch clamp experiments are performed using an Axopatch 200B amplifier (Axon Instruments, Burlingame, Calif.) linked to an IBM compatible personal computer equipped with pCLAMP software. Microelectrodes are filled with 3 M CsCl and have typical resistances from 0.5 to 2.5 MΩ. The external recording solution is 20 mM BaCl₂, 1 mM MgCl₂, 10 mM HEPES, 40 mM TEACl, 10 mM Glucose, 65 mM CsCl, (pH 7.2). The internal pipette solution is 105 mM CsCl, 25 mM TEACl, 1 mM CaCl₂, 11 mM EGTA, 10 mM HEPES (pH 7.2). Currents are typically elicited from a holding potential of −100 mV to various test potentials. Data are filtered at 1 kHz and recorded directly on the harddrive of a personal computer. Leak subtraction is carried out on-line using a standard P/5 protocol. Currents are analyzed using pCLAMP versions 5.5 and 6.0. Macroscopic current-voltage relations are fitted with the equation I={1/(1+exp(−(Vₘ−Vₕ)/S)}×G−(Vₘ−Eᵣₑᵥ), where Vₘ is the test potential, Vₕ is the voltage at which half of the channels are activated, and S reflects the steepness of the activation curve and is an indication of the effective gating charge movement. Inactivation curves are normalized to 1 and fitted with I=(1/l+exp ((Vₘ−Vₕ)/S) with Vₘ being the holding potential. Single channel recordings are performed in the cell-attached mode with the following pipette solution (in mM): 100 BaCl₂, 10 HEPES, pH 7.4 and bath solution: 100 KCl, 10 EGTA, 2 MgCl₂, 10 HEPES, pH 7.4.

B. Transient Transfection in Xenopus Oocytes

Stage V and VI Xenopus oocytes are prepared as described by Dascal et al (1986), Expression and modulation of voltage-gated calcium channels after RNA injection into Xenopus oocytes. Science 231:1147-1150. After enzymatic dissociation with collagenase, oocytes nuclei are microinjected with the human α_{1I} calcium channel cDNA expression vector construct (approximately 10 ng DNA per nucleus) using a Drummond nanoject apparatus. The human α_{1I} calcium channel may be injected alone, or in combination with other mammalian calcium channel subunit cDNAs, such as the α2-δ and β1b subunits. After incubation from 48 to 96 hrs macroscopic currents are recorded using a standard two microelectrode voltage-clamp (Axoclamp 2A, Axon Instruments, Burlingame, Calif.) in a bathing medium containing (in mM): 40 Ba(OH)₂, 25 TEA-OH, 25 NaOH, 2 CsOH, 5 HEPES (pH titrated to 7.3 with methan-sulfonic acid). Pipettes of typical resistance ranging from 0.5 to 1.5 mΩ are filled with 2.8M CsCl, 0.2M CsOH, 10 mM HEPES, 10 mM BAPTA free acid. Endogenous Ca (and Ba)—activated Cl currents are suppressed by systematically injecting 10-30 nl of a solution containing 100 mM BAPTA-free acid, 10 mM HEPES (pH titrated to 7.2 with CsOH) using a third pipette connected to a pneumatic injector. Leak currents and capacitive transients are subtracted using a standard P/5 procedure.

### EXAMPLE 3

### Construction of Stable Cell Lines Expressing Human α_{1I} Calcium Channels

Mammalian cell lines stably expressing human α_{1I} calcium channels are constructed by transfecting the α_{1I} calcium channel cDNA into mammalian cells such as HEK 293 and selecting for antibiotic resistance encoded for by an expression vector. Briefly, the full-length human α_{1I} calcium channel cDNA subcloned into a vertebrate expression vector with a selectable marker, such as the pcDNA3 (InvitroGen, San Diego, Calif.), is transfected into HEK 293 cells by calcium phosphate coprecipitation or lipofection or electroporation or other method according to well known procedures (Methods in Enzymology, Volume 185, Gene Expression Technology (1990) Edited by Goeddel, D. V.). The human α_{1I} calcium channel may be transfected alone, or in combination with other mammalian calcium channel subunit cDNAs, such as the α2-δ and β1b subunits, either in a similar expression vector or other type of vector using different selectable markers. After incubation for 2 days in nonselective conditions, the medium is supplemented with Geneticin (G418) at a concentration of between 600 to 800 ug/ml. After 3 to 4 weeks in this medium, cells which are resistant to G418 are visible and can be cloned as isolated colonies using standard cloning rings. After growing up each isolated colony to confluency to establish cell lines, the expression of human α_{1I} calcium channels can be determined at with standard gene expression methods such as Northern blotting, RNase protection and reverse-transcriptase PCR.

The functional detection of human α_{1I} calcium channels in stably transfected cells can be examined electrophysiologically, such as by whole patch clamp or single channel analysis (see above). Other means of detecting functional calcium channels include the use of radiolabeled ⁴⁵Ca uptake, fluorescence spectroscopy using calcium sensitive dyes such as FURA-2, and the binding or displacement of radiolabeled ligands that interact with the calcium channel.

1. An isolated DNA fragment comprising a sequence of nucleotides that encodes a calcium channel, wherein the sequence of nucleotides is selected from sequences of nucleotides encoding a protein including the sequence of amino acids set forth in SEQ ID. No. 19, and sequences of nucleotides that hybridize under non-stringent conditions to DNA encoding a protein including the sequence set forth in SEQ ID No. 19.

2. The DNA fragment of claim 1, wherein the sequence of nucleotides is selected from sequences of nucleotides encoding a protein including the sequence of amino acids set forth in SEQ ID. No. 18, and sequences of nucleotides that hybridize under non-stringent conditions to DNA encoding a protein including the sequence set forth in SEQ ID No. 18.

3. The DNA fragment of claim 1, wherein the calcium channel is a human neuronal calcium channel.

4. A vertebrate expression vector containing the DNA fragment of claim 1.

5. A vertebrate expression vector containing the DNA fragment of claim 2.

6. A eukaryotic cell transiently or stably transformed with the vertebrate expression vector of claim 4, said cell expressing the calcium channel encoded by the DNA fragment.

7. The eukaryotic cell of claim 6, wherein the cell is further transformed with and expresses an α2δ or a β calcium channel subunit, or both.

8. A eukaryotic cell transiently or stably transformed with a heterologous DNA fragment according to claim 1, said cell expressing the calcium channel encoded by the DNA fragment.

9. The eukaryotic cell of claim 8, wherein the cell is further transformed with and expresses an α2δ or a β calcium channel subunit, or both.

10. A method for the production of the α-_{1I} protein of an animal cell calcium channel comprising, culturing the cell of claim 6 under conditions whereby the DNA encoding the calcium channel subunit is expressed and the α-_{1I} subunit is produced.

11. A process for producing the eukaryotic cell that is transiently or stably transformed and expresses a calcium channel, comprising the step of introducing RNA or DNA having a sequence selected from among sequences that encode a protein including the sequence of amino acids set forth in SEQ ID. No. 19, and sequences of nucleotides that hybridize under non-stringent conditions to DNA encoding a protein including the sequence set forth in SEQ ID No. 19 and RNA or DNA encoding an α2δ or a β calcium channel subunit into a cell.

12. A method of identifying compounds capable of acting as agonists or antagonists for the α-_{1I} calcium channel, comprising contacting a cell according to claim 4 with an agent to be tested, and evaluating the interaction, if any, between the agent to be tested and the calcium channel.

13. An isolated DNA fragment comprising a sequence of nucleotides that encodes a human calcium channel subunit, wherein the sequence of nucleotides is selected from sequences of nucleotides including the sequence set forth in SEQ ID No. 17.

14. An isolated DNA fragment having the sequence given by SEQ ID No. 19.

15. A method for mapping the distribution of calcium channel subunits within a tissue sample comprising the steps of exposing the tissue to a reagent comprising a directly or indirectly detectable label coupled to a DNA fragment comprising a sequence selected from among those sequences given by SEQ ID Nos. 13-20, and detecting reagent that has bound to the tissue.


SEQUENCE LISTING (33 sequences)

SEQ ID NO: 1 - DNA, 24 bp, Rat
gtcaaaactcaggccttctactgg

SEQ ID NO: 2 - DNA, 24 bp, Rat
aacgtgttcttggctatcgcggtg

SEQ ID NO: 3 - DNA, 24 bp, Rat
gtgaaagcacagagcttctactgg

SEQ ID NO: 4 - DNA, 24 bp, Rat
aacgttttcttggccattgctgtg

SEQ ID NO: 5 - DNA, 24 bp, Rat
gttaaatccaacgtcttctactgg

SEQ ID NO: 6 - DNA, 24 bp, Rat
aatgtgttcttggccattgcggtg

SEQ ID NO: 7 - DNA, 24 bp, Rat
gtgaagtctgtcacgttttactgg

SEQ ID NO: 8 - DNA, 24 bp, Rat
aagctcttcttggccattgctgta

SEQ ID NO: 9 - DNA, 24 bp, Rat
gtcaagtcgcaagtgttctactgg

SEQ ID NO: 10 - DNA, 24 bp, Rat
aatgtattcttggctatcgctgtg

SEQ ID NO: 11 - DNA, 21 bp, Rat
atctaygcyrtsatyggsatg

SEQ ID NO: 12 - DNA, 20 bp, Rat
atggacaayttygastaytc

SEQ ID NO: 13 - DNA, 168 bp, Human
  Expressed sequence tag H55225
gtgatcactctggaaggctgggtggagatcatgtactacgtgatggatgctcactccttctacaacttcatctacttcatcctgcttatcatacccctcttgccttgcaccccatatggtcttcccagagtgagctcatccacctcgtcatgcctgactcgacgttca

SEQ ID NO: 14 - DNA, 98 bp, Human
  Expressed sequence tag H55617
gatggtcgagtactccctggaccttcagaacatcaacctgtcagccatccgcaccgtgcgcgtcctgaggcccctcaaagccatcaaccgcgtgccca

SEQ ID NO: 15 - DNA, 94 bp, Human
  Expressed sequence tag H55223
catgctggtgatcctgctgaactgcgtgacacttggcatgtaccagccgtgcgacgacatggactgcctgtccgaccgctgcaagatcctgcag

SEQ ID NO: 16 - DNA, 123 bp, Human
  Expressed sequence tag H55544
gtatctctggttactttagtagccaacactcttggctactcagaccttggtcccattaaatccctgcgaaccttgagagcactaagacctctaagagctttgtctagatttgaaggaatgagg

SEQ ID NO: 17 - DNA, 343 bp, Human
  Expressed sequence tag F07776
ttctctccattgtaggaatgtttctggctgaactgatagaaaagtattttgtgtgccctaccctgttncgagtgatccgtcttgccaggattggccgaatcctacgtctgatcaaaggagcaaaggggatccgcacgctgctctttgctttgatgatgtcccttcctgcgttgtttaacatcggnctccttcttttcctggtcatgttcatctacgncatctttgggatgtccaattttgcctatgttaagagggaagttgggatcgatgacatgttnanctttgagacctttggcaacagcatgatctgcctgttccaaattacaacctctgctggctggga

SEQ ID NO: 18 - DNA, 5562 bp, Human
  CDS (1)...(5562): Human alpha-1 partial sequence from BAC bK206c7
atgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggctctgaaacagctgatgttcaaattggtggccactgttgctcgaacacatgctacaccgtcacacatcacgggtggtcctggaacagggatgcacacgggcaccttccaggaaggagctgagcctggttcatctcagcaccctgaggcacaggccacgtatacagcagggtgcaccccagcccccacgggcgatcccacctgctgctttgtccttgacttggtgtgcacgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcaggcaacatcaacctgtcagccatccgcaccgtgcgcgtcctgaggcccctcaaagccatcaaccgcgtgcccagtatgcggatcctggtgaacctgctcctggacacactgcccatgctggggaatgtcctgctgctctgcttctttgtcttcttcatctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcataagtgagctcatccacctcgtcatgcctgactgcagcttcagcacagcacagtccccaaaatgtcaaggtgattcactcccaggagtcgctgctgaatccctgctgctgcgagactctagctcctcagtcatcactgatgaggctgcagccatggagaacctcctggcgggcacctccaagggggatgaaagctatctgctcaggctggccggcagccaagttcactcccaggctcagcaaatgctggggagggggctgggccctgaaagcctggaaactggagaggagccccactcgtggagccctcgggccacaaggagatgggatccccaatgccaaccagggcagcctctcccccttcatttcatgcaagcacaggtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccctctcacagtctgggaatcgattcttgggaggcaagcagaagaatgcacgctcagagctgccgcccacccgtcctcgggtgccagccatccaggcgtgggctcggaggaggccccagagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcaggccagtgcagcgcagccgggccgggcctgcgggagaggacaaaatccagacctttgcatgaccctcaaggccccttgtctctgtcacaacgtcccttcaccaggccagggtgtcctgtcccatccagtgactccaccccatacagccccatggcgcatggagacaggaaagcagggacacggatgtgaagaaggaccaggacaacgaagcagtgacatgtttgccctggagatgatcctgaagctggctgcatttgggctcttcgactacctgcgtaacccctacaacatcttcgacagcatcattgtcatcatcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggaggtgactgtggtcttggcagaggaagcacccccacagggcctgcgaaagactgggcgagggagaggtggcctggatgggggagggctgcaattcaaacttctagcaggcaacctatccctaaaggagggggttgctgatgaggtgggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctggtgggtggtcttagagccacagcgggggtgcaggctgcctttgggcacctggtgccccagccgtgggtgtgcctgtggggcgctgacccgaacgggaactccttccagtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacgtgagtgggggcggggccgaaggggacctgaccctgtgcttccgcgtccgcaagatgatcgacgtctataagcccgactggtgcgaggtccgcgaagactggtctgtctacctcttctctcccgagaacaggctcagggatctgggctgggtaagcctcgagtgccagggaaaggtgggtgacctcgtggtgtgggtgtatggtcagaggaggcagcgccagaccattattgcccacaaactcttcgactacgtcgtcctggccttcatctttctcaactgcatcaccatcgccctggagcggcctcagatcgaggccggcagcaccgaacgcatctttctcaccgtgtccaactacatcttcacggccatcttcgtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatcggcaacatcgtgctcatctgctgtgccttcttcatcatctttggcatcctgggagtgcagctcttcaagggcaagttctaccactgtctgggcgtggacacccgcaacatcaccaaccgctcggactgcatggccgccaactaccgctgggtccatcacaaatacaacttcgacaacctgggccaggctctgatgtccctctttgtcctggcatccaaggatggttgggtgaacatcatgtacaatggactggatgctgttgctgtggaccagcagcctgtgaccaaccacaacccctggatgctgctgtacttcatctccttcctgctcatcgtcagcttctttgtgctcaacatgtttgtgggtgtcgtggtggagaacttccacaagtgccggcagcaccaggaggctgaagaggcacggcggcgtgaggagaagcggctgcggcgcctggagaagaagcgccggaaggcccagcggctgccctactatgccacctattgtcacacccggctgctcatccactccatgtgcaccagccactacctggacatcttcatcaccttcatcatctgcctcaacgtggtcaccatgtccctggagcactacaatcagcccacg

SEQ ID NO: 19 - protein, 1853 aa, Human
FFVSANPWVSFTSFDLNVANMDNFFAPVFTMGKYYTQGDKVLMPLAIQALKQLMFKLVATVARTHATPSHITGGPGTGMHTGTFQEGAEPGSSQHPEAQATYTAGCTPAPTGDPTCCFVLDLVCTWFECVSMLVILLNCVTLGMYQPCDDMDCLSDRCKILQVFDDFIFIFFAMEMVLKMVALGIFGKKCYLGDTWNRLDFFIVMAGNINLSAIRTVRVLRPLKAINRVPSMRILVNLLLDTLPMLGNVLLLCFFVFFIFGIIGVQLWAGLLRNRCFLEENFTIQGDVALPPYYQPEEDDEMPFICSLSGDNGIMGCHEIPPLKEQGRECCLSKDDVYDFGAGRQDLNASGLCVNWNRYYNVCRTGSANPHKGAINFDNIGYAWIVIFQVITLEGWVEIMYYVMDAHSFYNFIYFILLIISELIHLVMPDCSFSTAQSPKCQGDSLPGVAAESLLLRDSSSSVITDEAAAMENLLAGTSKGDESYLLRLAGSQVHSQAQQMLGRGLGPESLETGEEPHSWSPRATRRWDPQCQPGQPLPLHFMQAQVGSFFMINLCLVVIATQFSETKQREHRLMLEQRQRYLSSSTVASYAEPGDCYEEIFQYVCHILRKAKRRALGLYQALQSRRQALGPEAPAPAKPGPHAKEPRHYPLTVWESILGRQAEECTLRAAAHPSSGASHPGVGSEEAPELCPQHSPLDATPHTLVQPIPATLASDPASCPCCQHEDGRRPSGLGSTDSGQEGSGSGSSAGGEDEADGDGARSSEDGASSELGKEEEEEEQADGAVWLCGDVWRETRAKLRGIVDSKYFNRGIMMAILVNTVSMGIEHHEQASAAQPGRACGRGQNPDLCMTLKAPCLCHNVPSPGQGVLSHPVTPPHTAPWRMETGKQGHGCEEGPGQRSSDMFALEMILKLAAFGLFDYLRNPYNIFDSIIVIISIWEIVGQADGGLSVLRTFRLLRVLKLVRFMPALRRQLVVLMKTMDNVATFCMLLMLFIFIFSILGMHIFGCKFSLRTDTGDTVPDRKNFDSLLWAIVTVFQILTQEDWNVVLYNGMASTSPWASLYFVALMTFGNYVLFNLLVAILVEGFQAEVTVVLAEEAPPQGLRKTGRGRGGLDGGGLQFKLLAGNLSLKEGVADEVGDANRSYSDEDQSSSNIEEFDKLQEGLDSSGDPKLCPIPMTPNGHLDPSLPLGGHLGPAGAAGPAPRLSLQPDPMLVALGSRKSSVMSLGRMSYDQRSLVGGLRATAGVQAAFGHLVPQPWVCLWGADPNGNSFQSSSRSSYYGPWGRSAAWASRRSSWNSLKHKPPSAEHESLLSAERGGGARVCEVAADEGPPRAAPLHTPHAHHVHHGPHLAHRHRHHRRTLSLDNRDSVDLAELVPAVGAHPRAAWRAAGPAPGHEDCNGRMPSIAKDVFTKMGDRGDRGEDEEEIDYVSGGGAEGDLTLCFRVRKMIDVYKPDWCEVREDWSVYLFSPENRLRDLGWVSLECQGKVGDLVVWVYGQRRQRQTIIAHKLFDYVVLAFIFLNCITIALERPQIEAGSTERIFLTVSNYIFTAIFVGEMTLKVVSLGLYFGEQAYLRSSWNVLDGFLVFVSIIDIVVSLASAGGAKILGVLRVLRLLRTLRPLRVISRAPGLKLVVETLISSLKPIGNIVLICCAFFIIFGILGVQLFKGKFYHCLGVDTRNITNRSDCMAANYRWVHHKYNFDNLGQALMSLFVLASKDGWVNIMYNGLDAVAVDQQPVTNHNPWMLLYFISFLLIVSFFVLNMFVGVVVENFHKCRQHQEAEEARRREEKRLRRLEKKRRKAQRLPYYATYCHTRLLIHSMCTSHYLDIFITFIICLNVVTMSLEHYNQPT

SEQ ID NO: 20 - DNA, 567 bp, Human
  CDS (1)...(567): Human alpha-1 partial sequence
atgcggatcctggtgaacctgctcctggacacactgcccatgctggggaatgtcctgctgctctgcttctttgtcttcttcacctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcagctttgacaacatcggttatgcttggattgtcatcttccaggtgatcactctggaaggctgggtggcgatcatgtactacgtgatggatgctctctccttctacaacttcgtctacttcatcctgcttatcata

SEQ ID NO: 21 - protein, 188 aa, Human
RILVNLLLDTLPMLGNVLLLCFFVFFTFGIIGVQLWAGLLRNRCFLEENFTIQGDVALPPYYQPEEDDEMPFICSLSGDNGIMGCHEIPPLKEQGRECCLSKDDVYDFGAGRQDLNASGLCVNWNRYYNVCRTGSANPHKGAISFDNIGYAWIVIFQVITLEGWVAIMYYVMDALSFYNFVYFILLII

SEQ ID NO: 22 - DNA, 567 bp, Rat
  CDS (1)...(567): Rat alpha-1 partial sequence
atgcggatcctggtgaacctgctgctcgacacgctgcccatgctggggaacgtgctcctgctctgtttcttcgtcttcttcatcttcggcatcattggcgtgcagctctgggcaggcctgctacggaaccgctgcttcctggaagaaaacttcaccatacaaggggatgtggccctgcccccttattaccaaccagaggaggatgacgagatgccctttatctgctccctgactggggacaatggcatcatgggctgccacgagatccccccactgaaggagcagggccgggaatgctgcctgtccaaagatgatgtgtatgacttcggggcggggcgccaggacctcaacgccagcggtctgtgcgtcaactggaaccgctactacaacgtctgccgcacgggcaacgccaaccctcacaagggcgccatcaactttgacaacattggctatgcctggattgtgattttccaggtgatcactctggaaggctgggtggagatcatgtactatgtgatggacgcacattctttctacaacttcatctacttcatcctgcttatcata

SEQ ID NO: 23 - protein, 188 aa, Rat
RILVNLLLDTLPMLGNVLLLCFFVFFIFGIIGVQLWAGLLRNRCFLEENFTIQGDVALPPYYQPEEDDEMPFICSLTGDNGIMGCHEIPPLKEQGRECCLSKDDVYDFGAGRQDLNASGLCVNWNRYYNVCRTGNANPHKGAINFDNIGYAWIVIFQVITLEGWVEIMYYVMDAHSFYNFIYFILLII

SEQ ID NO: 24 - protein, 31 aa, Human
VISLEGWTDIMYYVQDAHSFWNWIYFVLLIV

SEQ ID NO: 25 - protein, 31 aa, C. elegans
VITLEGWVEIMYYVMDAHSFYNFIYFILLII

SEQ ID NO: 26 - protein, 23 aa, Human
NINLTAIRTVRVLRPLRAVNRIP

SEQ ID NO: 27 - protein, 23 aa, C. elegans
NINLSAIRTVRVLRPLKAINRVP

SEQ ID NO: 28 - protein, 19 aa, Human
MAVIMINCVTLGMYRPCED

SEQ ID NO: 29 - protein, 19 aa, C. elegans
MLVILLNCVTLGMYQPCDD

SEQ ID NO: 30 - protein, 23 aa, Human
LTAIRTVRVLRPLRAVNRIPSMR

SEQ ID NO: 31 - protein, 23 aa, C. elegans
IKSLRTLRALRPLRALSRFEGMR

SEQ ID NO: 32 - protein, 41 aa, Human
PTIIRVMRVLRIARVLKLLKMAKGIRSLLDTVGEALPQVGN

SEQ ID NO: 33 - protein, 40 aa, C. elegans
  VARIANT (1)...(40): Xaa = Any Amino Acid
PTLXRVIRLARIGRILRLIKAKGIRTLLFALMMSLPALFN